(19) United States
(12) Patent Application Publication
(10) Pub. No.: US 2009/0216240 A1
(43) Pub. Date: Aug. 27, 2009

(54) SURGICAL INSERTION DEVICE FOR USE IN ORTHOPEDIC SURGERY

(75) Inventor: Gregory J. Posdal, South Barrington, IL (US)

Correspondence Address: MICHAEL P. MAZZA, LLC, 686 CRESCENT BLVD., GLEN ELYN, IL, 60137, US

(73) Assignee: SURGICAL INSTRUMENT SERVICE COMPANY, Carol Stream, IL (US)

(21) Appl. No.: 12/036,990

(22) Filed: Feb. 25, 2008

(51) Int. Cl. A61B 17/58

(52) U.S. Cl. 606/99

(57) ABSTRACT

A device, and a method for using it, for implanting a cup liner within a cup attached to the acetabulum of the hip bone, during hip replacement surgery. In a preferred embodiment, the device includes a main body with proximal and distal ends. The distal end may terminate in a liner seat and an engaging mechanism. Initially, the engaging mechanism may be coupled to the cup liner; a slight retraction of the engaging mechanism relative to the main body and liner seat may be sufficient to properly seat the cup liner on the liner seat. The device is now used to place the cup liner within the cup. The engaging mechanism may again be retracted to disengage the cup liner from the engaging mechanism, without dislodging the cup liner from the cup.

### BACKGROUND OF THE INVENTION

The present invention relates to an insertion device and method for facilitating hip replacement surgery. More specifically, the present relates to an insertion device and method for implanting a cup liner into a cup, which is itself seated within the acetabulum or cup socket of the hip/pelvic bone.

With modem hip replacement surgery, in summary fashion, the hip/pelvic bone is disclocated from the femur bone, and the femoral head (ball joint) is removed from the top of the femur bone. The cup portion or “acetabulum” which forms the resulting, exposed socket of the upper portion of the hip bone is then reamed, removing and smoothing worn portions. A prosthetic, metal cup with a non-smooth surface to stimulate bone growth, is then located within the acetabulum, and fixed in position using one or more screws. A cup liner with a smooth inside, concave surface is then seated within the cup. After reaming the femur bone, a broach or metal stem is then inserted into the femur, and a prosthetic ball is located on the end of the metal stem. The femoral ball is then inserted into the cup liner of the hip bone, completing the femoral/pelvic hip joint.

In practice, properly orienting and locating the cup liner within the cup covering the acetabulum of the hip bone, and then decoupling the cup liner from the insertion device, have proven to be problematic and time-consuming tasks. Of course, any time that may be saved during a surgical procedure is valuable. Sight lines may be nonexistent, requiring the procedure to occur based on feel. Even if the surgical team has appropriately sized the cup liner, if the cup liner is not located exactly correctly within the cup, it will need to be removed and re-located; cup liner removal can be problematic, as seen by FIG. 6A, as the cup liner can get wedged and stuck within the cup at an improper orientation. In the past, when improper seating occurs, the surgeon has used a hammer or mallet to vibrate the metal cup to facilitate liner removal. Many patients have a substantial amount of tissue (depth) between the implanted cup and the skin surface and it is difficult for the surgeon to gain entry to the cup liner. In practice, during surgery, it can take as long as 45 minutes to dislodge the cup liner.

For some years, hip replacement surgeons have requested an insertion device that would solve the problems discussed above. In the past few years, surgeons have utilized a planar (straight, not bent) insertion device with a suction cup on its distal tip; the suction cup did not have the ability to retract or extend relative to the main body of the device. Such prior insertion devices did not resolve the problematic issues of adequately engaging the cup liner, aligning the liner with the cup, and releasing the liner from a proximal release point.

### DEFINITION OF CLAIM TERMS

The terms used in the claims of the patent as filed are intended to have their broadest meaning consistent with the requirements of law. Where alternative meanings are possible, the broadest meaning is intended. All words used in the claims are intended to be used in the normal, customary usage of grammar and the English language.

### SUMMARY OF THE INVENTION

The objects mentioned above, as well as other objects, are solved by the present invention, which overcomes disadvantages of prior insertion devices and methods used for locating prosthetic cups in the hip cup socket, while providing new advantages not previously associated with such devices and methods.

In a preferred embodiment of the invention, a device is used to implant a cup liner within a cup attached to the acetabulum of the hip bone during hip replacement surgery. The preferred device includes a main body having proximal and distal ends. The distal end may terminate in an engaging mechanism that may be used to selectively engage with, and to selectively disengage from, the cup liner. Following seating of the cup liner within the cup, the engaging mechanism, such as a suction cup, may be retracted in a proximal direction relative to the main body to remove the engaging mechanism from the cup liner. Prior to coupling the engaging mechanism to the cup liner, the engaging mechanism may be extended in a distal direction relative to the main body. Selective disengagement of the cup liner is preferably and conveniently actuated at the proximal end of the device.

In a particularly preferred embodiment, a distal assembly located at the distal end of the device may include the engaging mechanism and a liner seat. The liner seat may be rigidly attached to the distal end. The engaging mechanism may be carried by, and movable within and relative to, the main body. The liner seat may have a circumferential edge sized to abut a circumferential edge of the cup liner. Preferably, mechanical interaction between the liner seat and the cup liner provides tactile feedback indicative as to when the cup liner is properly seated on the liner seat. The cup liner is preferably retained on the liner seat under tension, such as using spring or elastic band tension.

Following coupling of the engaging mechanism to the cup liner, and seating of the cup liner within the cup, the engaging mechanism may be retracted in a proximal direction relative to the main body, thereby dislodging the engaging mechanism from the liner seat, preferably in a substantially wobble-free manner, without dislodging the cup liner from its seating within the cup. To enable extension or retraction of the engaging mechanism relative to the main body, a portion of the main body may include a slot, and a slide handle may be positioned outside the main body and movable along the slot. In this embodiment, movement of the slide handle enables the engaging mechanism to move distally or proximally relative to the main body of the device.

In a preferred embodiment, the main body has a main axis, and the engaging mechanism moves along an engaging axis, and the main and engaging axes are not substantially aligned, and preferably form a substantial angle between them, such as 30-60 degrees, and most preferably about 45 degrees, to provide the instrument user with a direct view of the surgical site (i.e., with such a substantial angle, the main body will not impair the viewpoint from the proximal end of the device to the distal end of the device).

The proximal end of the device may be connected to the engaging mechanism using various mechanisms, such as a wire rope located within the main body and traveling along the main and engaging axes. A liner having a smooth inner surface may surround at least a portion of the wire rope, for example, to keep it from kinking during its movement along the bent or elbow portion of the preferred device.

In another preferred embodiment, a device may be provided to implant a cup liner within a cup attached to the acetabulum of the hip bone during hip replacement surgery. The device may include a main body having a main axis and proximal and distal ends. The distal end may terminate in an engaging mechanism movable along an engaging axis and designed to selectively engage with, and to selectively disengage from, the cup liner. Preferably in this embodiment, the main and engaging axes are not aligned, and selective disengagement of the engaging mechanism from the cup liner is actuated at the proximal end of the device. Preferably, the main and engaging axes form an angle of between about 30-60 degrees.

In still another alternative embodiment of the present invention, a method may be employed to use a device to implant a cup liner within a cup attached to the acetabulum of the hip bone during hip replacement surgery. The device may have a main body and proximal and distal ends. Following exposure of the acetabulum and attachment of the cup to the acetabulum, an engaging mechanism located at the distal end of the device may be coupled to the cup liner. The distal end may terminate in and/or be rigidly attached to a liner seat. Retracting the engaging mechanism in a proximal direction relative to the main body causes the cup liner to be seated on the liner seat. The device may now be manipulated to cause the cup liner to be seated within the cup. Further retracting the engaging mechanism in a proximal direction relative to the main body may now cause the engaging mechanism to be disengaged from the cup liner without disengaging the cup liner from the cup. The step of coupling the engaging mechanism to the cup liner may be preceded by the step of extending the engaging mechanism relative to the main body in a distal direction.

### BRIEF DESCRIPTION OF THE DRAWINGS

The novel features which are characteristic of the invention are set forth in the appended claims. The invention itself, however, together with further objects and attendant advantages thereof, will be best understood by reference to the following description taken in connection with the accompanying drawings. The drawings illustrate currently preferred embodiments of the present invention. As further explained below, it will be understood that other embodiments, not shown in the drawings, also fall within the spirit and scope of the invention.

FIG. 1 is perspective view of a surgical site concerning a hip replacement procedure;

FIG. 2 is a perspective view of the ball joint of the femur bone prior to insertion within the acetabulum of the hip/pelvic bone;

FIG. 3 is an exploded, parts perspective view of the implanted devices;

FIG. 4 is an enlarged, perspective view of the cup attached to the acetabulum;

FIG. 5 is a perspective view of the surgical insertion device of the present invention;

FIGS. 6A-6C are enlarged, partial perspective views of the distal end of the device of FIG. 5, showing the cup liner misaligned with (FIG. 6A) and aligned with (FIGS. 6B and 6C) the prosthetic cup attached to the acetabulum;

FIG. 7 is an exploded, perspective view showing the components of the preferred embodiment of the surgical instrument shown in FIG. 5; and

FIG. 8 is a cross-sectional view of the preferred embodiment of the surgical instrument shown in FIG. 5; and

FIGS. 8A and 8B are partial, enlarged cross-sectional views of proximal and distal portions, respectively, of the preferred embodiment of the surgical instrument shown in FIGS. 5 and 8.

The components in the drawings are not necessarily to scale, emphasis instead being placed upon clearly illustrating the principles of the present invention.

### DETAILED DESCRIPTION OF THE PREFERRED EMBODIMENTS

Set forth below is a description of what are currently believed to be the preferred embodiments and/or best examples of the invention claimed. Future and present alternatives and modifications to these preferred embodiments are contemplated. Any alternatives or modifications which make insubstantial changes in function, in purpose, in structure or in result are intended to be covered by the claims of this patent.

Below, “SIS” refers to Surgical Instrument Service Company of Carol Stream, Ill. “McMaster” refers to McMaster-Carr of Chicago, Ill. “Atlas Fibre” refers to Atlas Fibre Company of Skokie, Ill.

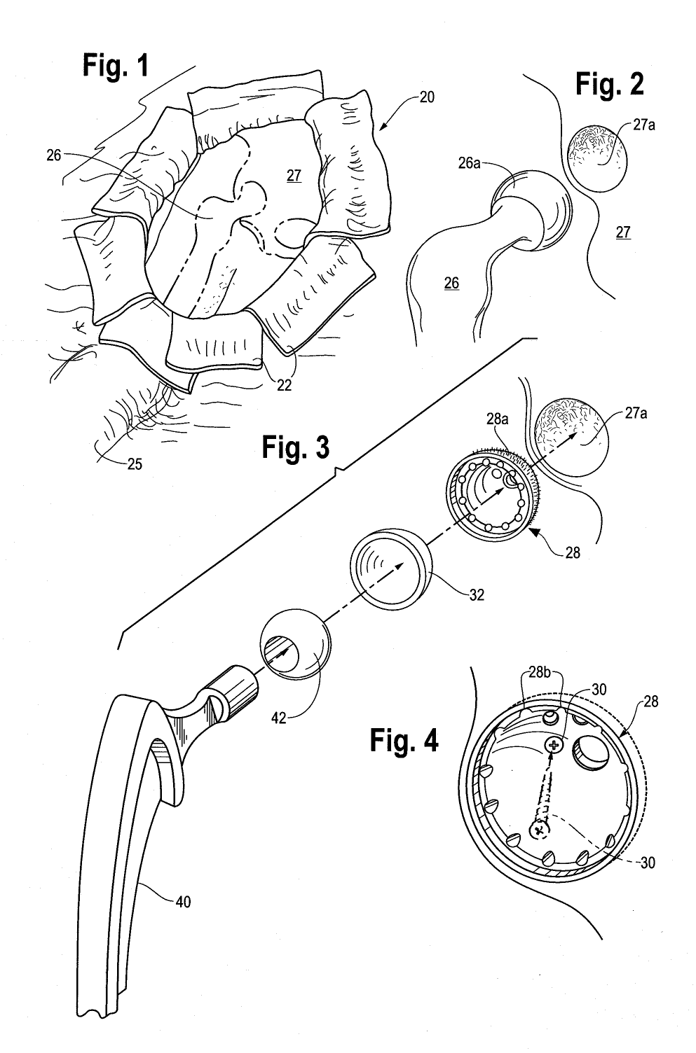

Referring first to FIGS. 1-2, a hip replacement surgical site, designated generally as 20 is shown, with retracted skin flaps 22, the femoral portion 25 of a leg, and the hip/pelvic bone portion 27. The upper portion of the femur bone 26 terminates in a ball portion 26a which attaches to the socket 27a of hip bone portion 27, as shown in FIG. 2.

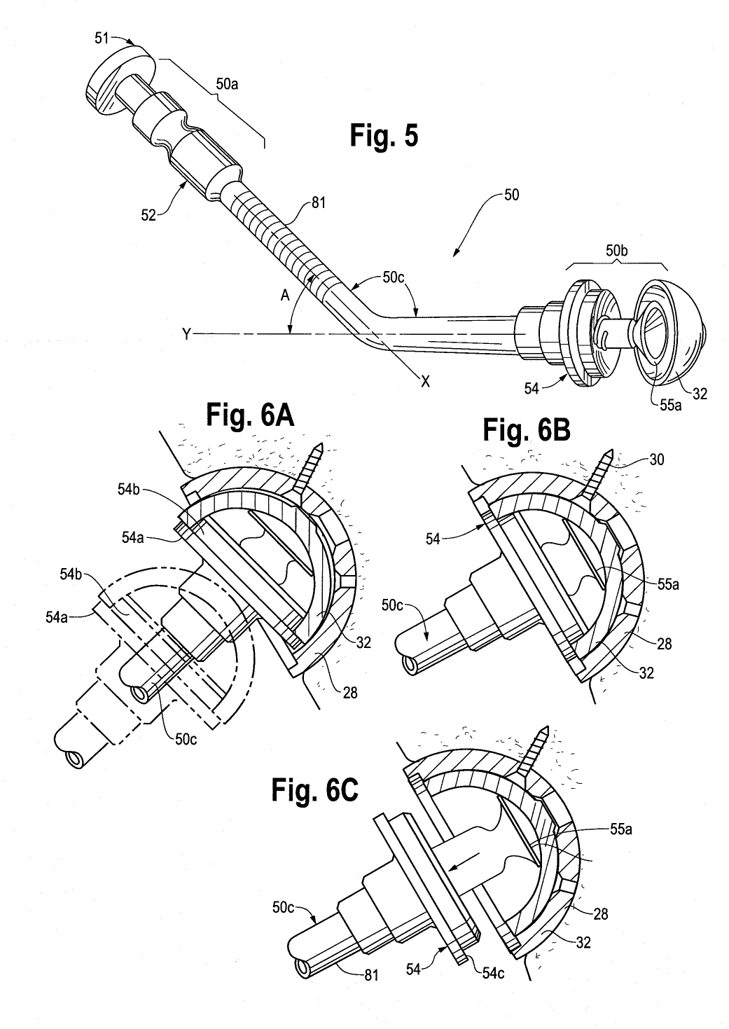

To provide an overview of the hip replacement surgery, after the femur has been dislocated from the hip, exposing the pelvic acetabulum/socket, the socket is reamed out to smooth worn portions, and a prosthetic cup 28 is fitted and attached to the acetabulum using screw(s) 30 (see FIGS. 4 and 6). For this purpose, the outer surface of cup 28 may include surface protrusions 28a useful in growing into the bone. Once the cup is seated within acetabulum 27a, liner removal notches 28b may facilitate cup removal if this is necessary. A cup liner 32 may be inserted into the inside, concave surface of cup 28 using an insertion device according to the present invention, as shown in FIGS. 5-6. (Cup liner 32 and ball 42 may be made of stainless steel, ceramic, plastic or another material.) A prosthetic stem 40 may be inserted into the femur bone, and a prosthetic ball 42 may be attached to the end of stem 40; ball 42 may then be fitted into cup liner 32, completing the femur/hip joint.

Referring now to FIG. 5, a preferred embodiment of the insertion device of the present invention, generally designated with numeral 50, is shown. Insertion device 50 has a proximal end 50a, a distal end 50b, and a main body 50c (e.g., 0.625-inch OD; 0.50-inch ID, machined by SIS, base tube available from McMaster and others). The tube 81 forming the main body of the device may be made of metal, preferably stainless steel, or possibly another material such as a hybrid or hard plastic, and may include gripping ridges 81a for handling convenience. Proximal end 50a may include a back handle 51 (e.g., 1.5-inch diameter, stainless steel 303, raw material available from McMaster) and a slide handle 52 (e.g., 1.0-inch diameter, Ultem (a heat-resistant, durable polymer), machined by SIS, raw material available from Atlas Fibre and others). Distal end 50b may include a liner seat 54 (e.g., 1.75-inch Ultem, machined by SIS, available from Atlas Fibre and others) and an engaging mechanism such as a suction cup 55a, which may be made of silicone.

Still referring to FIG. 5, in a particularly preferred embodiment, insertion device 50 may be angled such that the portion of the main body including proximal end 50a has a longitudinal axis along line “X” (“the main axis”) while the portion of the main body including distal end 50b has a longitudinal axis along line “Y” (“the engaging axis”). The main and engaging axes form an angle “A” which is preferably a substantial angle, i.e., about 30-60 degrees, and perhaps more preferably about 45 degrees, but which may have a different curvature, given the procedure, surgeon preference, etc. It is preferred to provide an insertion device that is bent, either in an angled or curved manner, for example, so that the device does not impede the surgeon's sight line during use. However, a straight device according to the principles of the invention, using an extendable and retractable engaging mechanism, will still provide substantial advantages over the prior art.

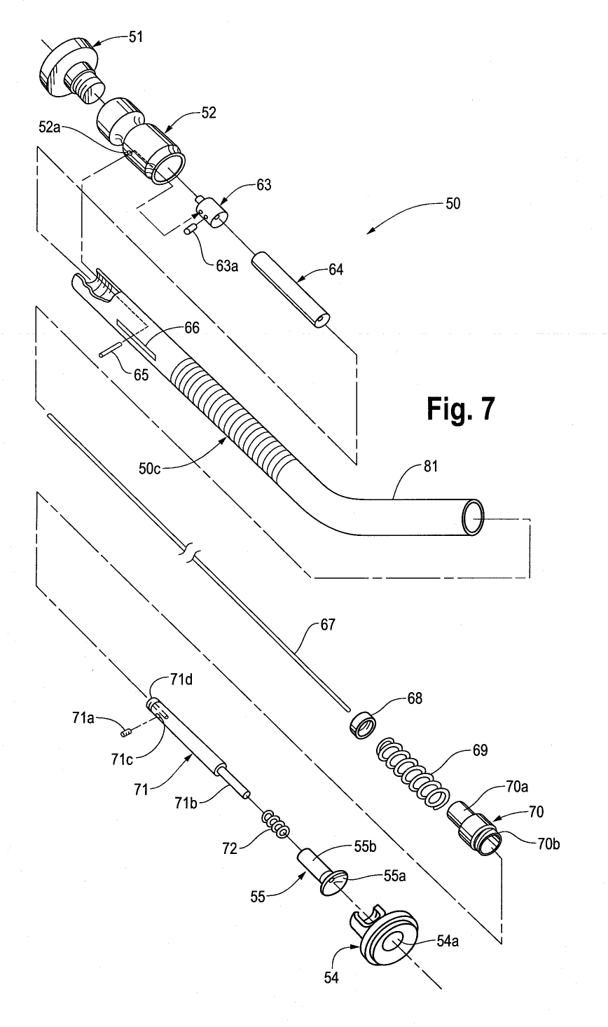

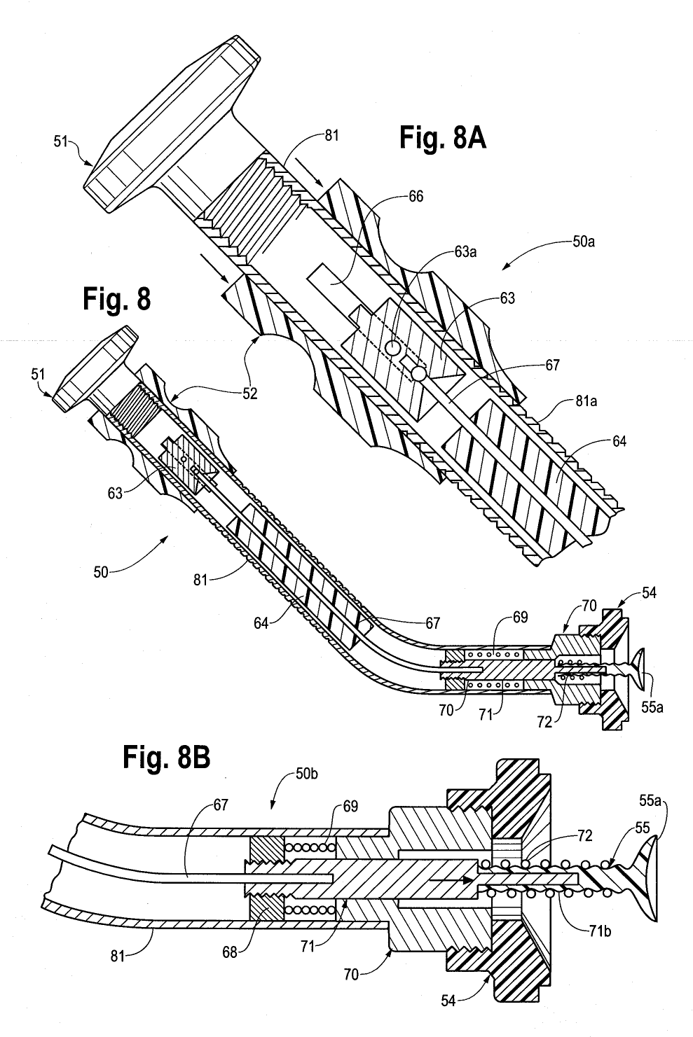

Referring now to FIGS. 7, 8 and 8A-8B, the components of the preferred surgical instrument 50 are shown. Starting at the proximal end, back handle 51 may be threadably attached to a proximal portion of the main body of instrument 50. Slide handle 52 may be fit over main body surface 81. Retention nut 63 (e.g., 0.5-inch diameter, brass, machined by SIS, raw material available from McMaster and others) may be sized to fit within the main body. Pin 65 (e.g., 303 SS, cut to size, raw material, McMaster) may pass through slot 66 on one side of the instrument body, then through aperture 52a of slide handle 52, then through aperture 63a of retention nut 63, and then through said corresponding components in reverse. In this manner, when pressure is exerted on slide handle 52, slide handle 52 may be slid a distance along, and relative to, main body 50c of instrument 50. Wire rope 67 (e.g., 1 by 19 (19 strands of wire), 303 SS, 0.100-inch OD) may be attached to retention nut 63 on a proximal end; wire rope may be attached to adjustment nut 68 (e.g., 303 SS, machined by SIS, raw material, McMaster). Instead of a wire rope, a thin stainless steel solid rod may be used, although given the angle it must traverse, this may not be preferred, due to potential kinking and/or strength issues.

Spring 69 (0.045-inch wire, fabricated by SIS) may be retained by adjustment nut 68 on one end, and may pass over a shaft portion of distal cup piston 71 (303 SS, machined by SIS, available from McMaster), and be retained by liner adaptor 70 on its other end. Liner adaptor 70 may threadably engage a distal portion of main body 50c on a proximal end, and may threadably engage liner seat 54 on a distal end.

Teflon liner/tube 64 (e.g., 0.75-inch OD, machined by SIS, available from McMaster, may be, e.g., 3.75-inches long) may be seated within a proximal section of main body 50c, such as just proximal of the elbow as shown in FIG. 8, to prevent the wire rope from kinking as it is slid within the angled portion of the instrument, as explained below.

Still referring to FIG. 7, distal cup piston 71 (e.g., 303 SS, machined by SIS, available from McMaster) may include a screw 71a which passes through aperture 71c to fix wire rope 67 in position. In the preferred embodiment, narrowed shaft 71b fits within tubular portion 55b of suction cup attachment 55, and is retained by spring 72. Suction cup attachment 55, including suction cup 55a, pass through aperture 54a of liner seat 54, so that the components of instrument 50 are finally arranged as shown in FIGS. 1 and 8.

In use, when instrument 50 is held at main body 50c and pressure is exerted on slide handle 52 toward distal end 50b (“distal pressure”), suction cup 55a will be moved distally relative to main body 50c and liner seat 54 a distance permitted by gap 66 in the main body, permitting suction cup 55a to engage cup liner 32 (see FIGS. 5 and 6A-6C). Now, if slide handle 52 is released, spring 69 will cause suction cup 55a to slightly retract in a proximal direction toward back handle 51, ensuring that if cup liner 32 has been properly engaged, that it will be properly seated against liner seat 54. During insertion, cup liner 32 may be oriented from an out-of-alignment position (e.g., FIG. 6A), into a properly aligned position (FIG. 6B), using the instrument; the surgeon or technician will feel a tactile feedback indicating proper alignment between liner seat 54 and cup liner 32 has occurred. The length of gap 66 and the spring tension are configured to provide sufficient retraction and retraction pressure such that the engaging mechanism maintains the cup liner properly seated against the liner seat, without providing too much pressure such that suction is lost (for example) between the engaging mechanism and the cup liner.

Liner seat 54 includes a post section 54a and a pillar section 54b preferably having a rounded edge; the narrower pillar section 54b terminates at its distal edge, leaving a gap region 54c on the outer and upper surface of post section 54a (see FIG. 6C). Cup liner 32 may be properly seated in this gap region, as shown in FIGS. 6B and 6C. When cup liner 32 is thus properly seated against liner seat 54, the instrument operator will feel this abutment, as the distal edge of cup liner 32 contacts gap region 54c on post section 54a. Now, the instrument may be used to seat cup liner 32 within cup 28 (FIG. 3); the instrument user will receive further tactile feedback that the cup liner has slid into and been appropriately seated within the cup. Liner seat may be made of a durable, autoclavable plastic such as Ultem, stainless steel, or other materials.

The preferred liner seat is appropriately sized to provide complete, 360° circumferential coverage for all edges of cup liner 32, such that the cup liner is seated on the liner seat in a substantially wobble-free manner (i.e., the cup liner will maintain substantially proper alignment, as shown in FIG. 6B, and not wobble or slide into misalignment, as shown in FIG. 6A). (Of course, small gaps in the circumferential edge of the liner seat will likely not materially impair cup liner seating in the cup.) Preferably, liner seat is not so large as to impair the view of the instrument user, so that he/she has a clear view of the cup and the cup liner during alignment and seating. To balance these competing interests, then, liner seat gap region 54c preferably is roughly the size of half of the thickness of the cup liner (leaving the other half of the thickness of the cup liner exposed and not abuting the liner seat).

Once cup liner is properly seated within cup 28, engaging mechanism may be removed. In the preferred embodiment, this may be accomplished by a single retracting pull on slide handle 52. This will disengage suction cup 55a from cup liner 32 without dislodging the proper seating of cup liner 32 from cup 28. An appropriate length of gap or slot 66, coupled with appropriate spring tension, will provide the user with the ability, after the cup liner is seated, to slightly retract the engaging mechanism, disengaging the cup liner from the engaging mechanism.

Referring again to FIG. 7, adjustment nut 68 may be threadably tightened or loosened on a threaded portion 71d of piston 71 to change the tension on spring 69, which will in turn change the engagement point at which the suction cup engages with, or disengages, from the cup liner.

In another alternative embodiment, instead of springs to provide the tension necessary between the engaging mechanism and the main body of the instrument, elastic bands may be used.

In a preferred embodiment, liner seat 54 may be easily disengaged from liner adaptor 70, such as by a threaded connection per threaded portion 70b. This enables liner seats and engaging mechanisms of various sizes to be easily changed on the instrument, depending upon the cup and cup liner size. Also, while in the preferred embodiment liner seat 54 and liner adaptor 70 are shown as separate components, these components may form one, integral component if desired; this may be desirable to allow metal stamping showing the sizing, for example.

In an exemplary embodiment, the combined length of portions 50a and 50C of instrument 50 up to the midpoint of the elbow may be about 7.75 inches, while the length of portion 50c distal of the mid-point of the elbow may be about 2.75 inches in length.

In a less preferred alternative embodiment, a spring need not be used. Thus, the instrument could be configured so that the engaging mechanism may be extended to be coupled to the cup liner, and then manually retracted without using spring tension; thereafter, following cup liner seating within the cup, the engaging mechanism may again be retracted to remove the engaging mechanism from the cup liner.

It will be understood that various modifications to the preferred embodiment disclosed above may be made. The above description is not intended to limit the meaning of the words used in the following claims that define the invention. Rather, it is contemplated that future modifications in structure, function or result will exist that are not substantial changes and that all such insubstantial changes are intended to be covered by the following claims.

We claim:

1. A device for use in implanting a cup liner within a cup attached to the acetabulum of the hip bone during hip replacement surgery, comprising:
a main body having proximal and distal ends, the distal end terminating in an engaging mechanism designed to selectively engage with, and to selectively disengage from, the cup liner;
wherein the engaging mechanism is coupled to the cup liner; and
following seating of the cup liner within the cup, the engaging mechanism is retracted in a proximal direction relative to the main body to remove the engaging mechanism from the cup liner.

2. The device of claim 1, wherein prior to coupling the engaging mechanism to the cup liner, the engaging mechanism is capable of being extended in a distal direction relative to the main body.

3. The device of claim 1, wherein selective disengagement of the cup liner is actuated at the proximal end of the device.

4. The device of claim 1, wherein the distal end terminates in a distal assembly comprising a liner seat and the engaging mechanism, and wherein the liner seat is rigidly attached to the distal end and the engaging mechanism is carried by, and movable within and relative to, the main body.

5. The device of claim 4, wherein the liner seat has a circumferential edge sized to abut a circumferential edge of the cup liner.

6. The device of claim 4, wherein following coupling of the engaging mechanism to the cup liner, the engaging mechanism is capable of being retracted in a proximal direction relative to the main body, thereby seating the cup liner on the liner seat.

7. The device of claim 5, following coupling of the engaging mechanism to the cup liner, the engaging mechanism is capable of being retracted in a proximal direction relative to the main body, thereby seating the cup liner on the liner seat in a substantially wobble-free manner.

8. The device of claim 4, wherein the cup liner is retained against the liner seat under tension.

9. The device of claim 4, wherein the cup liner is retained against the liner seat under spring tension.

10. The device of claim 1, wherein the main body has a main axis, and the engaging mechanism moves along an engaging axis, and wherein the main and engaging axes are not substantially aligned.

11. The device of claim 10, wherein the main and engaging axes form a substantial angle between them to provide the instrument user with a direct view of the surgical site.

12. The device of claim 11, wherein the main and engaging axes form an angle of between about 30-60 degrees.

13. The device of claim 10, wherein the main and engaging axes form an angle of about 45 degrees.

14. The device of claim 4, wherein mechanical interaction between the liner seat and the cup liner provides tactile feedback indicative of when the cup liner is properly seated on the liner seat.

15. The device of claim 4, wherein mechanical interaction between the liner seat and the cup liner provides tactile feedback indicative of when the cup liner is properly seated in the cup.

16. The device of claim 1, wherein the engaging mechanism comprises a suction cup.

17. The device of claim 1, further comprising means for connecting the proximal end of the device to the engaging mechanism.

18. The device of claim 17, wherein the connecting means comprises a wire rope located within the main body and along the main and engaging axes.

19. The device of claim 17, further comprising a liner having a smooth inner surface, the liner surrounding at least a portion of the connecting means.

20. The device of claim 1, wherein a portion of the main body includes a slot, and further comprising a slide handle movable along the slot.

21. The device of claim 1, wherein the proximal end includes a slidable handle, and wherein moving the slidable handle relative to the main body enables the engaging mechanism to move distally or proximally relative to the main body of the device.

22. A device for use in implanting a cup liner within a cup attached to the acetabulum of the hip bone during hip replacement surgery, comprising:
a main body having a main axis and proximal and distal ends, the distal end terminating in an engaging mechanism movable along an engaging axis and designed to selectively engage with, and to selectively disengage from, the cup liner;
wherein the main and engaging axes are not aligned, and wherein selective disengagement of the engaging mechanism from the cup liner is actuated at the proximal end of the device.

23. The device of claim 22, wherein the main and engaging axes form an angle of between about 30-60 degrees.

24. A method for using a device to implant a cup liner within a cup attached to the acetabulum of the hip bone during hip replacement surgery, the device having a main body and proximal and distal ends, comprising the steps of:
following exposure of the acetabulum and attachment of the cup to the acetabulum, coupling an engaging mechanism located at the distal end of the device to the cup liner, the distal end terminating in a liner seat;
retracting the engaging mechanism in a proximal direction relative to the main body, causing the cup liner to be seated on the liner seat;
manipulating the device to cause the cup liner to be seated within the cup;
further retracting the engaging mechanism in a proximal direction relative to the main body to cause the engaging mechanism to be disengaged from the cup liner without disengaging the cup liner from the cup.

25. The method of claim 24, wherein the step of coupling the engaging mechanism to the cup liner is preceded by the step of extending the engaging mechanism relative to the main body in a distal direction.
